(19) United States
(12) Patent Application Publication
(10) Pub. No.: US 2006/0225156 A1
(43) Pub. Date: Oct. 5, 2006

(54) Canola variety SW 013186

(75) Inventor: Bodil Jonsson, Lund (SE)

Correspondence Address: FULBRIGHT & JAWORSKI L.L.P., 600 CONGRESS AVE., SUITE 2400, AUSTIN, TX, 78701, US

(73) Assignee: SW SEED LDT

(21) Appl. No.: 11/388,765

(22) Filed: Mar. 24, 2006

Related U.S. Application Data
(60) Provisional application No. 60/666,505, filed on Mar. 30, 2005.

(51) Int. Cl. A01H 5/00; C12N 5/04

(52) U.S. Cl. 800/306; 435/419

(57) ABSTRACT

The invention relates to the novel canola variety designated SW 013186. Provided by the invention are the seeds, plants, plant parts and derivatives of the canola variety SW 013186. Also provided by the invention are tissue cultures of the canola variety SW 013186 and the plants regenerated therefrom. Still further provided by the invention are methods for producing canola plants by crossing the canola variety SW 013186 with itself or another canola variety and plants produced by such methods.

This application claims the priority of U.S. Provisional Patent Appl. Ser. No. 60/666,505, filed Mar. 30, 2005, the entire disclosure of which is specifically incorporated herein by reference.

### BACKGROUND OF THE INVENTION

1. Field of the Invention

The present invention relates to a novel rapeseed line designated SW 013186, as well as methods of use and derivatives thereof. Since such line is of high quality and possesses a relatively low level of erucic acid in the vegetable oil component and a relatively low level of glucosinolate content in the meal component, it can be termed “canola” in accordance with the terminology commonly used by plant scientists.

2. Description of Related Art

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include resistance to diseases and insects, tolerance to environmental stress, resistance to herbicides, improvements in seed oil composition and numerous other agronomic traits that may be desirable to the farmer or end user.

The goal of plant breeding is to develop new, unique and superior varieties. The breeder initially selects and crosses two or more parental lines, followed by repeated selfing and selection, producing many new genetic combinations. Each year, the plant breeder selects the germplasm to advance to the next generation. This germplasm is grown under unique and different geographical, climatic and soil conditions, and further selections are then made, during and at the end of the growing season. The varieties which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same variety twice by using the exact same original parents and the same selection techniques. This unpredictability results in the expenditure of large amounts of research monies to develop superior new canola varieties.

Choice of breeding or selection methods depends on the mode of plant reproduction, the heritability of the trait(s) being improved, and the type of variety used commercially (e.g., F₁ hybrid variety, pureline variety, etc.). For highly heritable traits, a choice of superior individual plants evaluated at a single location will be effective, whereas for traits with low heritability, selection should be based on mean values obtained from replicated evaluations of families of related plants. Popular breeding methods that have been used in canola include mass and recurrent selection, backcrossing, pedigree breeding and haploidy. One particularly efficient known method for breeding of canola varieties is pedigree breeding combined with doubled haploid production. General descriptions of such breeding techniques are well known in the art (see, e.g., Downey and Rakow, 1987: Rapeseed and Mustard. In: Fehr, W. R. (ed.), Principles of Cultivar Development, 437-486. New York: Macmillan and Co.; Thompson, 1983: Breeding winter oilseed rape Brassica napus. Advances in Applied Biology 7: 1-104; and Oilseed Rape, Ward et. al., Farming Press Ltd., Wharefedale Road, Ipswich, Suffolk (1985), each of which are hereby incorporated by reference).

The complexity of inheritance influences choice of the breeding method. Backcross breeding is used to transfer one or a few favorable genes for a highly heritable trait into a desirable variety. This approach has been used extensively for breeding disease-resistant plant varieties. Various recurrent selection techniques are used to improve quantitatively inherited traits controlled by numerous genes. The use of recurrent selection in self-pollinating crops depends on the ease of pollination, the frequency of successful hybrids from each pollination, and the number of offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful varieties produced per unit of input (e.g., per year, per dollar expended, etc.).

Promising advanced breeding lines are thoroughly tested and compared to appropriate standards in environments representative of the commercial target area(s) for generally three or more years. The best lines are candidates for new commercial varieties. Those still deficient in a few traits may be used as parents to produce new populations for further selection.

These processes, which lead to the final step of marketing and distribution, may take as much as eight to 12 years from the time the first cross is made. Therefore, development of new varieties is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to one or more widely grown standard varieties. Single observations are generally inconclusive, while replicated observations provide a better estimate of genetic worth.

Pureline cultivars, such as generally used in canola and many other crops, are commonly bred by hybridization of two or more parents followed by selection. The complexity of inheritance, the breeding objectives and the available resources influence the breeding method. The development of new varieties requires development and selection, the crossing of varieties and selection of progeny from superior crosses. The development of beneficial characteristics during such breeding may be aided by mutagenizing breeding stock during one or more generation(s) (see, e.g., Anderson (1995); Slade et al., (2004)).

Pedigree breeding is commonly used for the improvement of self-pollinating crops. Two parents which possess favorable, complementary traits are crossed to produce an F₁. An F₂ population is produced by selfing one or several F₁'s. Selection of the best individuals may begin in the F₂ population (or later depending upon the breeders objectives); then, beginning in the F₃, the best individuals in the best families can be selected. Replicated testing of families can begin in the F₃ or F₄ generation to improve the effectiveness of selection for traits with low heritability. At an advanced stage of inbreeding (i.e., F₆ and F₇), the best lines or mixtures of phenotypically similar lines are tested for potential release as new varieties.

Mass and recurrent selections can be used to improve populations of either self-or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

The modified single seed descent procedures involve harvesting multiple seed from each plant in a population and combining them to form a bulk. Part of the bulk is used to plant the next generation and part is put in reserve. This procedure has been used to save labor at harvest and to maintain adequate seed quantities of the population. The multiple-seed procedure may be used to save labor. The multiple-seed procedure also makes it possible to plant the same number of seeds of a population each generation of inbreeding. Enough seeds are harvested to make up for those plants that did not germinate or produce seed.

Descriptions of other breeding methods that are commonly used for different traits and crops can be found in one of several reference books (e.g., Allard, 1960; Simmonds, 1979; Sneep et al., 1979; Fehr, 1987a,b).

Proper testing should detect any major faults and establish the level of superiority or improvement over current varieties. In addition to showing superior performance, there must be a demand for a new variety that is compatible with industry standards or which creates a new market. The introduction of a new variety will incur additional costs to the seed producer, the grower, processor and consumer; for special advertising and marketing, altered seed and commercial production practices, and new product utilization. The testing preceding release of a new variety should take into consideration research and development costs as well as technical superiority of the final variety. For seed-propagated varieties, it must be feasible to produce seed easily and economically.

Canola is an important and valuable field crop. Thus, a continuing goal of plant breeders is to develop stable, high yielding canola varieties that are agronomically sound. To accomplish this goal, the canola breeder must select and develop plants that have the traits that result in superior cultivars. Among traits that may be deemed important are resistance to diseases and insects, tolerance to environmental stress, and improved agronomic traits. The breeder initially selects and crosses two or more parental lines, followed by generation advancement and selection, thus producing many new genetic combinations. The breeder can theoretically generate billions of different genetic combinations via this procedure.

Brassica napus canola plants, absent the use of sterility systems, are recognized to commonly be self-fertile with approximately 70 to 90 percent of the seed normally forming as the result of self-pollination. The percentage of cross pollination may be further enhanced when populations of recognized insect pollinators at a given growing site are greater. Thus open pollination is often used in commercial canola production. Currently, Brassica napus canola is being recognized as an increasingly important oilseed crop and a source of meal in many parts of the world. The oil as removed from the seeds commonly contains a lesser concentration of endogenously formed saturated fatty acids than other vegetable oils and is well suited for use in the production of salad oil or other food products or in cooking or frying applications. The oil also finds utility in industrial applications. Additionally, the meal component of the seeds can be used as a nutritious protein concentrate for livestock. “Canola” refers to rapeseed (Brassica) which as an erucic acid (C22:1) content of at most 2 percent by weight based on the total fatty acid content of a seed, preferably at most 0.5 percent by weight and most preferably essentially 0 percent by weight and which produces, after crushing, an air-dried meal containing less than 30 micromoles per gram of defatted (oil-free) meal. These types of seeds are distinguished by their edibility in comparison to more traditional varieties of the species.

### SUMMARY OF THE INVENTION

One aspect of the present invention relates to seed of the canola variety SW 013186. The invention also relates to plants produced by growing the seed of the canola variety SW 013186, as well as the derivatives of such plants. As used herein, the term “plant” includes plant cells, plant protoplasts, plant cells of a tissue culture from which canola plants can be regenerated, plant calli, plant clumps, and plant cells that are intact in plants or parts of plants, such as pollen, flowers, seeds, leaves, stems, and the like.

Another aspect of the invention relates to a tissue culture of regenerable cells of the canola variety SW 013186, as well as plants regenerated therefrom, wherein the regenerated canola plant expresses all the physiological and morphological characteristics of a plant grown from the canola seed designated SW 013186.

Yet another aspect of the current invention is a canola plant of the canola variety SW 013186 comprising at least a first transgene. In particular embodiments of the invention, a plant is defined as comprising a single locus conversion. The single locus may comprise a transgene gene which has been introduced by genetic transformation into a plant of the canola variety SW 013186 or a progenitor plant. In certain other embodiments of the invention, a dominant or recessive allele may be introduced. The locus conversion may confer potentially any desired trait upon the plant as described herein.

Still yet another aspect of the invention relates to a first generation (F₁) hybrid canola seed produced by crossing a plant of the canola variety SW 013186 to a second canola plant. Also included in the invention are the F₁ hybrid canola plants grown from the hybrid seed produced by crossing the canola variety SW 013186 to a second canola plant. Still further included in the invention are the seeds of an F₁ hybrid plant produced with the canola variety SW 013186 as one parent.

Still yet another aspect of the invention is a method of producing canola seeds comprising crossing a plant of the canola variety SW 013186 to any canola plant, including itself or another plant of the variety SW 013186. In particular embodiments of the invention, the method of crossing comprises the steps of a) planting seeds of the canola variety SW 013186; b) cultivating canola plants resulting from said seeds until said plants bear flowers; c) allowing fertilization of the flowers of said plants; and, d) harvesting seeds produced from said plants.

Still yet another aspect of the invention is a method of producing hybrid canola seeds comprising crossing the canola variety SW 013186 to a second plant which is nonisogenic to the canola variety SW 013186. In particular embodiments of the invention, the crossing comprises the steps of a) planting seeds of canola variety SW 013186 and a second, distinct canola plant, b) cultivating the canola plants grown from the seeds until the plants bear flowers; c) cross pollinating a flower on one of the two plants with the pollen of the other plant, and d) harvesting the seeds resulting from the cross pollinating.

Still yet another aspect of the invention is a method for developing a canola plant in a canola breeding program comprising: obtaining a canola plant, or its parts, of the variety SW 013186; and b) employing said plant or parts as a source of breeding material using plant breeding techniques. In the method, the plant breeding techniques may be selected from the group consisting of recurrent selection, mass selection, bulk selection, backcrossing, pedigree breeding, genetic marker-assisted selection and genetic transformation. In certain embodiments of the invention, the canola plant of variety SW 013186 is used as the male or female parent.

Still yet another aspect of the invention is a method of producing a canola plant derived from the canola variety SW 013186, the method comprising the steps of: (a) preparing a progeny plant derived from canola variety SW 013186 by crossing a plant of the canola variety SW 013186 with a second canola plant; and (b) crossing the progeny plant with itself or a second plant to produce a progeny plant of a subsequent generation which is derived from a plant of the canola variety SW 013186. In one embodiment of the invention, the method further comprises: (c) crossing the progeny plant of a subsequent generation with itself or a second plant; and (d) repeating steps (b) and (c) for at least 2-10 additional generations to produce an inbred canola plant derived from the canola variety SW 013186.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DETAILED DESCRIPTION OF THE INVENTION

The invention provides, in one aspect, methods and composition relating to plants, seeds and derivatives of the canola variety SW 013186. The canola variety SW 013186 has been judged to be uniform for breeding purposes and testing. The variety can be reproduced by planting and growing seeds of the variety under self-pollinating or sib-pollinating conditions, as is known to those of skill in the agricultural arts. Variety SW 013186 shows no variants other than what would normally be expected due to environment or that would occur for almost any characteristic during the course of repeated sexual reproduction.

Canola variety SW 013186 exhibits superior traits including RoundUp Ready® and winter hardiness. The line is adapted and performs consistently in Southern Kansas and Oklahoma, Southern Nebraska, Western Arkansas. However the yield of SW 013186 is lower than the local conventional check varieties, Wichita and Sumner. The results of an objective description of the variety are presented below, in Table 1. Those of skill in the art will recognize that these are typical values that may vary due to environment and that other values that are substantially equivalent are within the scope of the invention.

TABLE 1
Phenotypic (Physiological and Morphological) Description of Variety SW 013186

| | |
|---|---|
| Pollen formation: | Normal. |
| Type: | Winter growth habit |
| Time of Flowering: | ˜178 days, Medium-Late, similar to Wichita and Sumner |
| Time of Bolting: | Medium, earlier than Wichita and Sumner |
| Flower Petal Coloration: | Medium yellow, circular petals, medium large in size and |
| | overlapping. |
| Maturity: | Medium to Medium-Late |
| Disease Reaction: | Moderately resistant to blackleg (Phoma) |
| Seed Coat Color: | Black. |
| Seed Size: | ˜3.66 mg, Medium |
| Oil Content: | Average, similar to Wichita and Sumner |
| Protein Content: | Below Average, lower than Wichita and Sumner |
| Plant Height: | ˜95 cm, Medium-Tall, similar to Sumner, shorter than Wichita |
| Resistance to Lodging: | Above Average, similar to Wichita and Sunmer |
| Canola Quality: | Average, Glucosinolate similar to Wichita and Sumner |
| Leaf Lobes: | Medium number. |
| Leaf Dentation: | Medium depth with serrations of largely rounded margin type, |
| | dentation depth is deeper than Wichita |
| Leaf Color: | Medium dark green. |
| Leaf Length: | Medium |
| Leaf Width: | Medium |
| Leaf Petiole: | Medium-Short length |
| Silique(pod) characteristics: | attached at a horizontal attitude, siliques are medium length (longer |
| | than Wichita) and, medium width (narrower than Wichita), with |
| | medium pedicel length and medium-long beak length (longer beak |
| | length than Wichita). |
| Winter Hardiness | Above average, similar to Wichita and Sumner |
| Herbicide Resistance: | Resistant to Glyphosate, RoundUp Ready ® |

The performance characteristics of canola variety SW 013186 were also analyzed and comparisons were made with competing varieties. Characteristics examined included RoundUp Ready®, winter hardiness, height, lodging, early bolting and early flower. The results of the analysis are presented below, in Tables 2-3.

TABLE 2-3
PERFORMANCE DATA FOR VARIETY SW 013186 AND SELECTED VARIETIES
2-YEAR DATA SUMMARY OF PRIVATE WINTER CANOLA TRIALS

| | | | | | | | |
|---|---|---|---|---|---|---|---|
| Herbicide | Yield¹ | Rel.² | Winter³ | Bolting⁴ | Flower⁵ | Height⁶ | |
| Tolerance | kg/ha | yield | Survival % | early % | early % | cm. | Lodging⁷ % |

Table 2 Comparison to Check variety Wichita

| | | | | | | | | |
|---|---|---|---|---|---|---|---|---|
| Wichita | Conventional | 3196 | 100 | 74.0 | 22.4 | 34.1 | 100 | 23.2 |
| SW 013186 | Roundup Ready ® | 2786* | 87 | 72.9 ns | 64.0*** | 53.0 ns | 95* | 57.5 ns |
| # Locations | | 9 | 9 | 8 | 6 | 5 | 5 | 2 |

Table 3 Comparison to Check variety Sumner

| | | | | | | | | |
|---|---|---|---|---|---|---|---|---|
| Sumner | Conventional | 3007 | 100 | 75.3 | 39.9 | 52.2 | 98.4 | 28.8 |
| SW 013186 | Roundup Ready ® | 2687* | 89 | 72.9 ns | 64.0** | 53.0 ns | 95.4 ns | 57.5 ns |
| # Locations | | 8 | 8 | 8 | 6 | 5 | 5 | 2 |

Locations Yield: Marianna, AR (03); Hutchenson, KS (03); Lahoma, Okla (03/04); Chickaha, Okla (03/04); Perkins, Okla (03/04); Haskel, Oka (04). Locations Agronomic: Oklahoma State University locations - Lahoma, Okla (03/04); Chickaha, Okla (03/04); Perkins, Okla (03/04); Haskel, Okla (04
¹Yield measured in kg/ha basis 10% moisture content
²Relative yield compared to the check variety(s) located on the top line of each table ie. Wichita, Sumner.
³Winter survival computed from plant count data in the spring after new season growth has resumed divided by the plant count data from the fall prior to the winter season multiplied by 100 to give a percentage survival
⁴Bolting early %. This refers to the plants in the spring which are shooting up stems with apical bud clusters just prior to the appearance of first flowers, divided by the total number of plants in the plot multiplied by 100 to give a percentage bolting. Elongation is from a strong rosette to the normal plant height and is observed approximately 170 days after fall sowing.
⁵Flower early %. This refers to the plants which have at least one open flower divided by the total number of plants in the plot multiplied by 100 to give a percentage flower. First flowers begin appearing approximately 175-178 days after fall sowing in these Southern locations ie. Oklahoma.
⁶Height in cm is recorded as the mean of multiple observations per replicate. Measurement if from the surface of the ground to the tip of the outstretched apical raceme.
⁷Lodging % is recorded at physiological maturity as the number of plants which are leaning over or fallen down from the basal attachment point divided by the total number of plants in the plot multiplied by 100.

I. Breeding Canola Variety SW 013186

One aspect of the current invention concerns methods for crossing the canola variety SW 013186 with itself or a second plant and the seeds and plants produced by such methods. These methods can be used for propagation of the canola variety SW 013186, or can be used to produce hybrid canola seeds and the plants grown therefrom. A hybrid plant can be used as a recurrent parent at any given stage in a backcrossing protocol during the production of a single locus conversion of the canola variety SW 013186.

The variety of the present invention is well suited to the development of new varieties based on the elite nature of the genetic background of the variety. In selecting a second plant to cross with SW 013186 for the purpose of developing novel canola varieties, it will typically be desired to choose those plants that themselves exhibit one or more selected desirable characteristics. Examples of potentially desired characteristics include resistance to diseases and insects, tolerance to environmental stress, improved agronomic traits and improved oil composition. Techniques for development of a new variety using one or more starting varieties are well known in the art and are described herein above. One particularly efficient method known for breeding of canola varieties is pedigree breeding combined with doubled haploid production.

Any time the canola variety SW 013186 is crossed with another, different, variety, first generation (F₁) canola progeny are produced. The hybrid progeny are produced regardless of characteristics of the two varieties produced. As such, an F₁ hybrid canola plant may be produced by crossing SW 013186 with any second canola plant. The second canola plant may be genetically homogeneous (e.g., inbred) or may itself be a hybrid. Therefore, any F₁ hybrid canola plant produced by crossing canola variety SW 013186 with a second canola plant is a part of the present invention.

Canola plants can be crossed by either natural or mechanical techniques. Natural pollination occurs in canola either by self pollination or natural cross pollination, which typically is aided by pollinating organisms. In either natural or artificial crosses, flowering and flowering time are an important consideration.

II. Improvement of Canola Varieties

In certain further aspects, the invention provides plants modified to include at least a first desired trait. Such plants may, in one embodiment, be developed by a plant breeding technique called backcrossing, wherein essentially all of the desired morphological and physiological characteristics of a variety are recovered in addition to a genetic locus transferred into the hybrid via the backcrossing technique. The term backcrossing as used herein refers to the repeated crossing of a hybrid progeny back to a starting variety into which introduction of the desired trait is being carried out. The parental plant which contributes the locus or loci for the desired trait is termed the nonrecurrent or donor parent. This terminology refers to the fact that the nonrecurrent parent is used one time in the backcross protocol and therefore does not recur.

The parental canola plant to which the locus or loci from the nonrecurrent parent are transferred is known as the recurrent parent as it is used for several rounds in the backcrossing protocol (Poehlman et al., 1995; Fehr, 1987; Sprague and Dudley, 1988). In a typical backcross protocol, the original line of interest (recurrent parent) is crossed to a second variety (nonrecurrent parent) that carries the genetic locus to be transferred. The resulting progeny from this cross are then crossed again to the recurrent parent and the process is repeated until a canola plant is obtained wherein essentially all of the desired morphological and physiological characteristics of the recurrent parent are recovered in the converted plant, in addition to the transferred locus from the nonrecurrent parent.

The backcross process may be accelerated by the use of genetic markers, such as Simple Sequence Length Polymorphisms (SSLPs) (Williams et al., 1990), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., 1998) to identify plants with the greatest genetic complement from the recurrent parent.

The selection of a suitable recurrent parent is an important step for a successful backcrossing procedure. The goal of a backcross protocol is to add or substitute one or more new traits in a variety. To accomplish this, a genetic locus of the recurrent parent is modified or substituted with the desired locus from the nonrecurrent parent, while retaining essentially all of the rest of the desired genetic, and therefore the desired physiological and morphological constitution of the original plant. The choice of the particular nonrecurrent parent will depend on the purpose of the backcross; one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many traits have been identified that are not regularly selected for in the development of a new variety but that can be improved by backcrossing techniques. A genetic locus conferring the traits may or may not be transgenic. Examples of such traits known to those of skill in the art include, but are not limited to, male sterility, herbicide resistance, resistance for bacterial, fungal, or viral disease, insect resistance, male fertility and enhanced nutritional quality. These genes are generally inherited through the nucleus, but may be inherited through the cytoplasm.

Direct selection may be applied where a genetic locus acts as a dominant trait. An example of a dominant trait is the herbicide resistance trait. For this selection process, the progeny of the initial cross are sprayed with the herbicide prior to the backcrossing. The spraying eliminates any plants which do not have the desired herbicide resistance characteristic, and only those plants which have the herbicide resistance gene are used in the subsequent backcross. This process is then repeated for all additional backcross generations.

Many useful traits are those which are introduced by genetic transformation techniques. Numerous methods for plant transformation have been developed, including biological and physical, plant transformation protocols (See, e.g., Miki et al., “Procedures for Introducing Foreign DNA into Plants” in Methods in Plant Molecular Biology and Biotechnology; and Glick, 1988).

Agrobacterium mediated transformation in particular is an efficient method for transformation of Brassica species. A number of protocols for Agrobacterium transformation that may find use in connection with the current variety have been described in the art (see, e.g., EP 0 116 718; EP 0 270 882; U.S. Pat. No. 5,750,871; U.S. Pat. No. 5,463,174; U.S. Pat. No. 5,188,958; Horsch, et al., (1985). Radke et al., (1992); Fry et al., (1987); Ohlsson and Eriksson, (1988); and Radke et al., (1988).

An advantage of Agrobacterium-mediated transfer is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

Another useful technique for transforming Brassica species is microprojectile bombardment (see, for example, U.S. Pat. Nos. 5,204,253 and 6,051,756; and Chen et al, 1994). For microprojectile bombardment, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for microprojectile bombardment is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

Still other types of transformation procedures known in the art include that may find use for plant transformation are electroporation, direct DNA uptake by protoplasts, sonication, microinjection, pollen-tube pathway mediated transformation, silicon carbon mediated transformation, plastid transformation (U.S. Pat. No. 6,515,206), and spheroplast-mediated transformation (see, e.g., Rakoczy-Trojanowska, 2002; Maliga, 2004; Zhang et al., 1994.).

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner. Protoplasts may also be employed for electroporation transformation of plants (Bates, 1994; Lazzeri, 1995). For example, the generation of transgenic cotyledon-derived protoplasts was described by Dhir and Widholm in Intl. Patent Appl. Publ. No. WO 92/17598. In addition to electroporation, transformation of plant protoplasts can be achieved using methods based on calcium phosphate precipitation, polyethylene glycol treatment, and combinations of these treatments (see, e.g., Potrykus et al., 1985; Omirulleh et al., 1993; Fromm et al., 1986; Uchimiya et al., 1986; Marcotte et al., 1988). Non-limiting examples of traits that may be introduced directly into a plant by such techniques, as well as by plant breeding techniques, are presented below.

A. Male Sterility

Pollination control systems and effective transfer of pollen from one parent to the other may be used to produce hybrid canola seed and plants. Male sterility genes can increase the efficiency with which hybrids are made, in that they eliminate the need to physically emasculate the plant used as a female in a given cross. Where one desires to employ male-sterility systems, it may be beneficial to also utilize one or more male-fertility restorer genes. For example, where cytoplasmic male sterility (CMS) is used, hybrid crossing requires three inbred lines: (1) a cytoplasmically male-sterile line having a CMS cytoplasm; (2) a fertile inbred with normal cytoplasm, which is isogenic with the CMS line for nuclear genes (“maintainer line”); and (3) a distinct, fertile inbred with normal cytoplasm, carrying a fertility restoring gene (“restorer” line). The CMS line is propagated by pollination with the maintainer line, with all of the progeny being male sterile, as the CMS cytoplasm is derived from the female parent. These male sterile plants can then be efficiently employed as the female parent in hybrid crosses with the restorer line, without the need for physical emasculation of the male reproductive parts of the female parent.

The presence of a male-fertility restorer gene results in the production of fully fertile F₁ hybrid progeny. If no restorer gene is present in the male parent, male-sterile hybrids are obtained. Examples of male-sterility genes and corresponding restorers which could be employed with the plants of the invention are well known to those of skill in the art of plant breeding. For example, the ogura cytoplasmic male sterility (cms) system, developed via protoplast fusion between radish (Raphanus sativus) and rapeseed (Brassica napus) is a frequently used method of hybrid production. It provides stable expression of the male sterility trait and an effective nuclear restorer gene (Ogura 1968; Pelletier et al., 1983; Heyn 1976). In developing new hybrid varieties, breeders may use self-incompatible (SI), cytoplasmic male sterile (CMS) and nuclear male sterile (NMS) Brassica plants as the female parent. When hybridization is conducted without using SI, CMS or NMS plants, it may be more difficult to obtain and isolate the desired traits in the progeny (F1 generation) because the parents are capable of undergoing both cross-pollination and self-pollination.

A fertility restorer for ogura cytoplasmic male sterile plants has been transferred to Brassica. The restorer gene, Rfl, in particular has been described, as have improved versions, See, e.g., WO 92/05251 and WO98/27806. Other sources of CMS sterility in canola include the Polima cytoplasmic male sterile plant, as well as those of U.S. Pat. No. 5,789,566. Still further examples are described in U.S. Pat. No. 5,973,233; WO97/02737; EP patent application 0 599042A; U.S. Pat. No. 6,229,072; and U.S. Pat. No. 4,658,085, each of the disclosures of which are specifically incorporated herein by reference.

B. Herbicide Resistance

Numerous herbicide resistance genes are known and may be employed with the plants of the invention. An example is a gene conferring resistance to a herbicide that inhibits the growing point or meristem, such as imidazolinone or sulfonylurea. Exemplary genes in this category code for mutant ALS and AHAS enzymes as described, for example, by Lee et al., (1988); Gleen et al., (1992); Miki et al., (1990).

Resistance genes for glyphosate (resistance conferred by mutant 5-enolpyruvl-3 phosphikimate synthase (EPSP) and aroA genes) and other phosphono compounds such as glufosinate (phosphinothricin acetyl transferase (PAT) and Streptomyces hygroscopicus phosphinothricin-acetyl transferase (bar) genes) may also be used. For example, U.S. Pat. No. 4,940,835 to Shah, et al., discloses the nucleotide sequence of a form of EPSPS that confers glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a phosphinothricin-acetyltransferase gene is provided in European application No. 0 242 246 to Leemans et al. DeGreef et al., (1989), describe the production of transgenic plants that express chimeric bar genes coding for phosphinothricin acetyl transferase activity. Exemplary genes conferring resistance to herbicidal phenoxy propionic acids and cycloshexones, such as sethoxydim and haloxyfop are the Acct-S1, Accl-S2 and Acct-S3 genes described by Marshall et al., (1992).

Genes are also known conferring resistance to a herbicide that inhibits photosynthesis, such as triazine (psbA and gs+ genes) and benzonitrile (nitrilase gene). Przibilla et al., (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., (1992).

Methods for transformation avoid use of antibiotic selection in plant transformation process have also been described, such as the use of the enzyme phosphomannose isomerase (PMI) encoded by the manA gene from E. coli (see, e.g., Miles et al., 1984). Other types of markers that may be used include heightened levels of tryptophan (Trp) and the marker D-amino acid oxidase.

C. Disease Resistance

Plant defenses are often activated by specific interaction between the product of a disease resistance gene (R) in the plant and the product of a corresponding avirulence (Avr) gene in the pathogen. A plant line can be transformed with cloned resistance gene to engineer plants that are resistant to specific pathogen strains. See, for example Jones et al., (1994) (cloning of the tomato Cf-9 gene for resistance to Cladosporium fulvum); Martin et al., (1993) (tomato Pto gene for resistance to Pseudomonas syringae pv.); Mindrinos et al., (1994) (Arabidopsis RSP2 gene for resistance to Pseudomonas syringae). Logemann et al., (1992), for example, disclose transgenic plants expressing a barley ribosome-inactivating gene have an increased resistance to fungal disease.

A viral-invasive protein or a complex toxin derived therefrom may also be used for viral disease resistance. For example, the accumulation of viral coat proteins in transformed plant cells imparts resistance to viral infection and/or disease development effected by the virus from which the coat protein gene is derived, as well as by related viruses. See Beachy et al., (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus.

A virus-specific antibody may also be used. See, for example, Tavladoraki et al., (1993), who show that transgenic plants expressing recombinant antibody genes are protected from virus attack.

D. Insect Resistance

One example of an insect resistance gene includes a Bacillus thuringiensis protein, a derivative thereof, or a synthetic polypeptide modeled thereon. See, for example, Geiser et al., (1986), who disclose the cloning and nucleotide sequence of a Bt δ-endotoxin gene. Moreover, DNA molecules encoding δ-endotoxin genes can be purchased from the American Type Culture Collection, Manassas, Va., for example, under ATCC Accession Nos. 40098, 67136, 31995 and 31998. Another example is a lectin. See, for example, Van Damme et al., (1994), who disclose the nucleotide sequences of several Clivia miniata mannose-binding lectin genes. A vitamin-binding protein may also be used, such as avidin. See PCT application US93/06487, the contents of which are hereby incorporated by reference. This application teaches the use of avidin and avidin homologues as larvicides against insect pests.

Yet another insect resistance gene is an enzyme inhibitor, for example, a protease or proteinase inhibitor or an amylase inhibitor. See, for example, Abe et al, (1987) (nucleotide sequence of rice cysteine proteinase inhibitor), Huub et al., (1993) (nucleotide sequence of cDNA encoding tobacco proteinase inhibitor I), and Sumitani et al., (1993) (nucleotide sequence of Streptomyces nitrosporeus α-amylase inhibitor). An insect-specific hormone or pheromone may also be used. See, for example, Hammock et al., (1990) disclosing baculovirus expression of cloned juvenile hormone esterase, an inactivator of juvenile hormone.

Still other examples include an insect-specific antibody or an immunotoxin derived therefrom and a developmental-arrestive protein. See Taylor et al., (1994), who described enzymatic inactivation in transgenic tobacco via production of single-chain antibody fragments.

E. Modified Fatty Acid, Phytate and Carbohydrate Metabolism

Genes may be used conferring modified fatty acid metabolism and thereby alter seed oil composition. For example, stearyl-ACP desaturase genes may be used. See Knutzon et al., (1992). Various fatty acid desaturases have also been described, such as a Saccharomyces cerevisiae OLE1 gene encoding delta-9 fatty acid desaturase, an enzyme which forms the monounsaturated palmitoleic (16:1) and oleic (18:1) fatty acids from palmitoyl (16:0) or stearoyl (18:0) CoA (McDonough et al., 1992); a gene encoding a stearoyl-acyl carrier protein Δ9 desaturase from castor (Fox et al., 1993); Δ6 and Δ12 desaturases from the cyanobacteria Synechocystis responsible for the conversion of linoleic acid (18:2) to gamma-linolenic acid (18:3 gamma) (Reddy et al., 1993); a gene from Arabidopsis thaliana that encodes an omega-3 desaturase (Arondel et al., 1992); plant Δ9 desaturases (PCT Application Publ. No. WO 91/13972) and soybean and Brassica Δ15 desaturases (European Patent Application Publ. No. EP 0616644). Expression of any one of these genes with a promoter functional in seeds may thereby used to modify seed oil composition in a canola plant.

Phytate metabolism may also be modified by introduction of a phytase-encoding gene to enhance breakdown of phytate, adding more free phosphate to the transformed plant. For example, see Van Hartingsveldt et al., (1993), for a disclosure of the nucleotide sequence of an Aspergillus niger phytase gene. This, for example, could be accomplished by cloning and then reintroducing DNA associated with the single allele which is responsible for mutants characterized by low levels of phytic acid. See Raboy et al, (1990).

A number of genes are known that may be used to alter carbohydrate metabolism. For example, plants may be transformed with a gene coding for an enzyme that alters the branching pattern of starch. See Shiroza et al., (1988) (nucleotide sequence of Streptococcus mutants fructosyltransferase gene), Steinmetz et al., (1985) (nucleotide sequence of Bacillus subtilis levansucrase gene), Pen et al., (1992) (production of transgenic plants that express Bacillus lichenifonnis α-amylase), Elliot et al., (1993) (nucleotide sequences of tomato invertase genes), Sergaard et al., (1993) (site-directed mutagenesis of barley α-amylase gene), and Fisher et al, (1993) (maize endosperm starch branching enzyme II). The Z10 gene encoding a 10 kD zein storage protein from maize may also be used to alter the quantities of 10 kD Zein in the cells relative to other components (Kirihara et al., 1988).

III. Tissue Cultures and In Vitro Regeneration of Canola Plants

A further aspect of the invention relates to tissue cultures of the canola variety designated SW 013186. As used herein, the term “tissue culture” indicates a composition comprising isolated cells of the same or a different type or a collection of such cells organized into parts of a plant. Exemplary types of tissue cultures are protoplasts, calli and plant cells that are intact in plants or parts of plants, such as embryos, pollen, flowers, leaves, roots, root tips, anthers, and the like. In a preferred embodiment, the tissue culture comprises embryos, protoplasts, meristematic cells, pollen, leaves or anthers. Exemplary procedures for tissue culture of canola plants are well known in the art and are further described herein (see, e.g., Chuong et al. (1985); Barsby et al., 1996); Kartha et al., (1974); Narasimhulu et al., 1988); Swanson, (1990); Swanson, (1989).

An important ability of a tissue culture is the capability to regenerate fertile plants. This allows, for example, transformation of the tissue culture cells followed by regeneration of transgenic plants. For transformation to be efficient and successful, DNA must be introduced into cells that give rise to plants or germ-line tissue.

Plants typically are regenerated via two distinct processes; shoot morphogenesis and somatic embryogenesis. Shoot morphogenesis is the process of shoot meristem organization and development. Shoots grow out from a source tissue and are excised and rooted to obtain an intact plant. During somatic embryogenesis, an embryo (similar to the zygotic embryo), containing both shoot and root axes, is formed from somatic plant tissue. An intact plant rather than a rooted shoot results from the germination of the somatic embryo.

Shoot morphogenesis and somatic embryogenesis are different processes and the specific route of regeneration is primarily dependent on the explant source and media used for tissue culture manipulations. While the systems are different, both systems show variety-specific responses where some lines are more responsive to tissue culture manipulations than others. A line that is highly responsive in shoot morphogenesis may not generate many somatic embryos. Lines that produce large numbers of embryos during an induction step may not give rise to rapidly-growing proliferative cultures. Therefore, it may be desired to optimize tissue culture conditions for each canola line. These optimizations may readily be carried out by one of skill in the art of tissue culture through small-scale culture studies. In addition to line-specific responses, proliferative cultures can be observed with both shoot morphogenesis and somatic embryogenesis. Proliferation is beneficial for both systems, as it allows a single, transformed cell to multiply to the point that it will contribute to germ-line tissue.

Embryogenic cultures can also be used successfully for regeneration, including regeneration of transgenic plants, if the origin of the embryos is recognized and the biological limitations of proliferative embryogenic cultures are understood. Biological limitations include the difficulty in developing proliferative embryogenic cultures and reduced fertility problems (culture-induced variation) associated with plants regenerated from long-term proliferative embryogenic cultures. Some of these problems are accentuated in prolonged cultures. The use of more recently cultured cells may decrease or eliminate such problems.

IV. Definitions

In the description and tables which follow, a number of terms are used. In order to provide a clear and consistent understanding of the specification and claims, the following definitions are provided:

A. Definition of Plant Characteristics

Anther Arrangement: The general disposition of the anthers in typical fully opened flowers is observed.

Anther Dotting: The level of anther dotting when the flowers are fully opened is observed.

Bolting Early %: The number of plants in the spring which are shooting up stems with apical bud clusters (elongation) just prior to the appearance of first flowers, divided by the total number of plants in the plot multiplied by 100 to give a percentage bolting. Elongation is from a strong rosette to the normal plant height and is observed approximately 170 days after fall sowing.

Chlorophyll Content: The typical chlorophyll content of the mature seeds is determined by using methods recommended by the WCC/RRC and is considered to be low if <8 ppm, medium if 8 to 15 ppm, and high if 15 to 30 ppm.

Cotyledon Length: The distance between the indentation at the top of the cotyledon and the point where the width of the petiole is approximately 4 mm.

Cotyledon Width: The width at the widest point of the cotyledon when the plant is at the two to three-leaf stage of development (mean of 50).

Cotyledon: A cotyledon is a type of seed leaf; a small leaf contained on a plant embryo. A cotyledon contains the food storage tissues of the seed. The embryo is a small plant contained within a mature seed.

Disease Resistance: Resistant to various diseases is evaluated and is expressed on a scale of 0 highly resistant, 5=highly susceptible. The WCC/RRC blackleg classification is based on % severity index described as follows: 0-30%=Resistant; 30%-50%=Moderately Resistant; 50%-70%=Moderately Susceptible; 70%-90%=Susceptible; >90%=Highly susceptible.

Fatty Acid Content: The typical percentages by weight of fatty acids present in the endogenously formed oil of the mature whole dried seeds are determined. During such determination the seeds are crushed and are extracted as fatty acid methyl esters following reaction with methanol and sodium methoxide. Next the resulting ester is analyzed for fatty acid content by gas liquid chromatography using a capillary column which allows separation on the basis of the degree of unsaturation and fatty acid chain length. This procedure is described in Daun et al., (1983), which is incorporated herein by reference.

Flower Bud Location: A determination is made whether typical buds are disposed above or below the most recently opened flowers.

Flower Early %. For winter type canola this refers to the plants which have at least one open flower divided by the total number of plants in the plot multiplied by 100 to give a percentage flower. First flowers begin appearing approximately 175-178 days after fall sowing in these Southern locations ie. Oklahoma

Flower Petal Coloration: The coloration of open exposed petals on the first day of flowering is observed.

Glucosinolate Content: The total glucosinolates of seed at 8.5% moisture as measured by AOCS Official Method AK-1-92 (Determination of glucosinolates content in rapeseed-colza by HPLC) is expressed micromoles per gram. Capillary gas chromatography of the trimethylsityl derivatives of extracted and purified desulfoglucosinolates with optimization to obtain optimum indole glucosinolate detection as described in “Procedures of the Western Canada Canola/Rapeseed Recommending Committee Incorporated for the Evaluation and Recommendation for Registration of Canola/Rapeseed Candidate Cultivars in Western Canada.”

Herbicide Resistance: Resistance to various herbicides when applied at standard recommended application rates is expressed on a scale of 1 (resistant), or 2 (susceptible).

Leaf Anthocyanin Coloration: The presence or absence of leaf anthocyanin coloration and the degree thereof if present are observed when the plant has reached the 9 to 11 leaf-stage.

Leaf Attachment to Stem: The presence or absence of clasping where the leaf attaches the stem, and when present the degree thereof are observed.

Leaf Attitude: The disposition of typical leaves with respect to the petiole is observed when at least 6 leaves of the plant are formed.

Leaf Color: The leaf blade coloration is observed when at least 6 leaves of the plant are completely developed.

Leaf Dentation: The margins of the upper stem leaves are observed for the presence or absence of indentation or serration, and the degree thereof if present when at least 6 leaves of the plant are completely developed.

Leaf Glaucousity: The presence or absence of a fine whitish powdery coating on the surface of the leaves, and the degree thereof when present are observed.

Leaf Lobes: The fully developed upper stem leaves are observed for the presence or absence of leaf lobes when at least 6 leaves of the plant are completely developed.

Leaf Length: The length of the leaf blades and petioles are observed when at least 6, leaves of the plant are completely developed (mean of 50).

Leaf Margin Hairiness: The leaf margins of the first leaf are observed for the presence or absence of pubescence, and the degree thereof when the plant is at the two leaf-stage.

Leaf Surface: The leaf surface is observed for the presence or absence of wrinkles when at least 6 leaves of the plant are completely developed.

Leaf Tip Reflexion: The presence or absence of bending of typical leaf tips and the degree thereof, if present are observed at the 6 to 11 leaf-stage.

Leaf Upper Side Hairiness: The upper surfaces of the leaves are observed for the presence or absence of hairiness, and the degree thereof if present when at least 6 of the leaves of the plant are formed.

Leaf Width: The width of the leaf blades are observed when at least 6 leaves of the plant are completely developed (mean of 50).

Length of Beak: The typical length of the silique beak when mature is observed and is expressed on a scale of 1 (short) to 5 (long).

Maturity: The number of days from planting to maturity is observed with maturity being defined as the plant stage when pods with seed color change, occurring from green to brown or black, on the bottom third of the pod bearing area of the main stem.

Number of Leaf Lobes: The frequency of leaf lobes when present is observed when at least 6 leaves of the plant are completely developed.

Oil Content: The typical percentage by weight oil present in the mature whole dried seeds is determined by ISO 10565:1993 Oilseeds Simultaneous determination of oil and water—Pulsed NMR method: Also, oil could be analyzed using NIR (Near Infra Red spectroscopy) as long as the instrument is calibrated and certified by Grain Research Laboratory of Canada.

Pedicel Length: The typical length of the silique peduncle when mature is observed and is expressed on a scale of 1 (short) to 5 (long).

Petal Length: The lengths of typical petals of fully opened flowers are observed (mean of 50).

Petal Width: The widths of typical petals of fully opened flowers are observed (mean of 50).

Petiole Length: The length of the petioles is observed in a line forming lobed leaves when at least 6 leaves of the plant are completely developed.

Plant Height: The overall plant height at the end of flowering is observed (mean of 50).

Pod Anthocyanin Coloration: The presence or absence at maturity of silique anthocyanin coloration, and the degree thereof if present are observed.

Pod Habit: The typical manner in which the silique are borne on the plant at maturity is observed.

Pod Length: The typical silique length is observed and is expressed on a scale of I (short) to 5 (long).

Pod Type: The overall configuration of the silique is observed.

Pod Width: The typical silique width when mature is observed and is expressed on a scale of 1 (narrow) to 5 (wide).

Pollen Formation: The relative level of pollen formation is observed at the time of dehiscence.

Protein Content: The typical percentage by weight of protein in the oil free meal of the mature whole dried seeds is determined by AOCS Official Method Ba 4e-93 Combustion Method for the Determination of Crude Protein. Also, protein could be analyzed using NIR (Near Infra Red spectroscopy) as long as the instrument is calibrated and certified by Grain Research Laboratory of Canada.

Resistance to Shattering: Resistance to silique shattering is observed at seed maturity and is expressed on a scale of 1 (poor) to 9 (excellent).

Resistant to Lodging: Lodging % is recorded at physiological maturity as the number of plants which are leaning over or fallen down from the basal attachment point divided by the total number of plants in the plot multiplied by 100.

Root Anthocyanin Coloration: The presence or absence of anthocyanin coloration in the skin at the top of the root is observed when the plant has reached at least the 6 leaf-stage.

Root Anthocyanin Expression: When anthocyanin coloration is present in skin at the top of the root, it further is observed for the exhibition of a reddish or bluish cast within such coloration when the plant has reached at least the 6 leaf-stage.

Root Anthocyanin Streaking: When anthocyanin coloration is present in the skin at the top of the root, it further is observed for the presence or absence of streaking within such coloration when the plant has reached at least the 6 leaf-stage.

Root Chlorophyll Coloration: The presence or absence of chlorophyll coloration in the skin at the top of the root is observed when the plant has reached at least the 6 leaf-stage.

Root Coloration Below Ground: The coloration of the root skin below ground is observed when the plant has reached at least the 6 leaf-stage.

Root Depth in Soil: The typical root depth is observed when the plant has reached at least the 6 leaf-stage.

Root Flesh Coloration: The internal coloration of the root flesh is observed when the plant has reached at least the 6 leaf-stage.

Seed Coat Color: The seed coat color of typical mature seeds is observed.

Seed Coat Mucilage: The presence or absence of mucilage on the seed coat is determined and is expressed on a scale of 1 (absent) to 9 (heavy). During such determination a petri dish is filled to a depth of 0.3 cm. with tap water provided at room temperature. Seeds are added to the petri dish and are immersed in water where they are allowed to stand for five minutes. The contents of the petri dish containing the immersed seeds next is examined under a stereo microscope equipped with transmitted light. The presence of mucilage and the level thereof is observed as the intensity of a halo surrounding each seed.

Seed Size: The weight in grams of 1,000 typical seeds is determined at maturity while such seeds exhibit a moisture content of approximately 5 to 6 percent by weight.

Seedling Growth Habit: The growth habit of young seedlings is observed for the presence of a weak (1) or strong (9) rosette character and is expressed on a scale of 1 to 9.

Seeds Per Pod: The average number of seeds per pod is observed (mean of 50).

Stem Anthocyanin Coloration: The presence or absence of leaf anthocyanin coloration and the intensity thereof if present are observed when the plant has reached the 9 to 11 leaf-stage.

Speed of Root Formation: The typical speed of root formation is observed when the plant has reached the 4 to 11 leaf-stage.

Time of Flowering: A determination is made of the number of days when at least 50 percent of the plants have one or more open buds on a terminal raceme in the year of sowing.

Type: This refers to whether the new line is considered to be primarily a Spring or Winter type of canola.

Winter Survival (Winter Type Only): Computed from plant count data in the spring after new season growth has resumed divided by the plant count data from the fall multiplied by 100 to give a percentage survival.

B. Additional Definitions

A: When used in conjunction with the word “comprising” or other open language in the claims, the words “a” and “an” denote “one or more.”

Allele: Any of one or more alternative forms of a gene locus, all of which alleles relate to one trait or characteristic. In a diploid cell or organism, the two alleles of a given gene occupy corresponding loci on a pair of homologous chromosomes.

Backcrossing: A process in which a breeder repeatedly crosses hybrid progeny, for example a first generation hybrid (F₁), back to one of the parents of the hybrid progeny. Backcrossing can be used to introduce one or more single locus conversions from one genetic background into another.

Crossing: The mating of two parent plants.

Cross-pollination: Fertilization by the union of two gametes from different plants.

Desired Agronomic Characteristics: Agronomic characteristics (which will vary from crop to crop and plant to plant) such as yield, maturity, pest resistance and oil composition which are desired in a commercially acceptable crop or plant.

Disease Resistance: The ability of plants to restrict the activities of a specified pest, such as an insect, fungus, virus, or bacterial.

Disease Tolerance: The ability of plants to endure a specified pest (such as an insect, fungus, virus or bacteria) or an adverse environmental condition and still perform and produce in spite of this disorder.

Donor Parent: The parent of a variety which contains the gene or trait of interest which is desired to be introduced into a second variety.

Emasculate: The removal of plant male sex organs or the inactivation of the organs with a cytoplasmic or nuclear genetic factor conferring male sterility or a chemical agent.

Essentially all the physiological and morphological characteristics: A plant having essentially all the physiological and morphological characteristics means a plant having the physiological and morphological characteristics, except for the characteristics derived from the desired trait.

F₁ Hybrid: The first generation progeny of the cross of two nonisogenic plants.

Genotype: The genetic constitution of a cell or organism.

Haploid: A cell or organism having one set of the two sets of chromosomes in a diploid.

Linkage: A phenomenon wherein alleles on the same chromosome tend to segregate together more often than expected by chance if their transmission was independent.

Phenotype: The detectable characteristics of a cell or organism, which characteristics are the manifestation of gene expression.

Recurrent Parent: The repeating parent (variety) in a backcross breeding program. The recurrent parent is the variety into which a gene or trait is desired to be introduced.

Regeneration: The development of a plant from tissue culture.

Self-pollination: The transfer of pollen from the anther to the stigma of the same plant or a plant of the same genotype.

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic locus comprising a sequence which has been introduced into the genome of a canola plant by transformation.

V. Deposit Information

Applicant has made a deposit of at least 2500 seeds of canola variety SW 013186 disclosed herein with the American Type Culture Collection (ATCC), 10801 University Boulevard, Manassas, Va. 20110-2209 USA under ATCC Accession No. PTA-______. The seeds were deposited with the ATCC on ______, and were taken from a deposit maintained since prior to the filing date of this application. Access to this deposit will be available during the pendency of the application to the Commissioner of Patents and Trademarks and persons determined by the Commissioner to be entitled thereto upon request. The deposit will be maintained for a period of 30 years, or 5 years after the most recent request, or for the enforceable life of the patent, whichever is longer, and will be replaced if it becomes nonviable during that period. Applicant does not waive any infringement of their rights granted under this patent or under the Plant Variety Protection Act (7 U.S.C. 2321 et seq.).

### REFERENCES

The following references, to the extent that they provide exemplary procedural or other details supplementary to those set forth herein, are specifically incorporated herein by reference.
U.S. Pat. No. 4,658,085
U.S. Pat. No. 4,769,061
U.S. Pat. No. 4,810,648
U.S. Pat. No. 4,940,835
U.S. Pat. No. 4,975,374
U.S. Pat. No. 5,188,958
U.S. Pat. No. 5,204,253
U.S. Pat. No. 5,463,174
U.S. Pat. No. 5,750,871
U.S. Pat. No. 5,789,566
U.S. Pat. No. 5,973,233
U.S. Pat. No. 6,051,756
U.S. Pat. No. 6,229,072
U.S. Pat. No. 6,515,206
Abe et al., J. Biol. Chem., 262:16793, 1987.
Allard, In: Principles of plant breeding, John Wiley & Sons, NY, University of California, Davis, Calif., 50-98, 1960.
Anderson, Methods Cell Biol., 48:31-58, 1995.
Arondel et al. Science, 258(5086):1353-1355, 1992.
Barsby et al., Plant Cell Reports, 5:101, 1986.
Bates, Mol. Biotechnol., 2(2):135-145, 1994.
Beachy et al., Ann. Rev. Phytopathol., 28:451, 1990.
Chen et al., Theor. Appl. Genet., 88:187-192, 1994.
Chuong et al. Plant Cell Reports, 4:4-6, 1985.
Daun et al., J. Amer. Oil Chem. Soc., 60:1751-1754, 1983.
DeGreef et al., Bio/Technology, 7:61, 1989.
Downey and Rakow, In: Principles of Cultivar Development, Fehr (Ed.), NY, Macmillan and Co., 437-486, 1987.
Elliot et al., Plant Molec. Biol., 21:515, 1993.
Eur. Appln. 0116718
Eur. Appln. 0 242 246
Eur. Appln. 0 270 882
Eur. Appln. 0 333 033
Eur. Appln. 0 599042A
Eur. Appln. 534 858
Eur. Appln. EP 0616644
Fehr, In: Principles of variety development, Theory and Technique (Vol 1) and Crop Species Soybean (Vol 2), Iowa State Univ., Macmillian Pub. Co., NY, 360-376, 1987b.
Fehr, In: Soybeans: Improvement, Production and Uses,” 2d Ed., Manograph 16:249, 1987a.
Fisher et al., Plant Physiol., 102:1045, 1993.
Fox et al. Proc. Natl. Acad. Sci. USA, 90(6):2486-2490, 1993.
Fromm et al., Nature, 319(6056):791-793, 1986.
Fry et al., Plant Cell Reports, 6:321-325, 1987.
Gleen et al., Plant Molec. Biology, 18:1185-1187, 1992.
Glick, Biotechnol. Fats and Oils Ind., 43-46, 1988.
Hammock et al., Nature, 344:458, 1990.
Hayes et al., Biochem. J. 285:173, 1992.
Heyn, Cruciferae Newsletter, 1: 15-16, 1976.
Horsch, et al., Science, 227:1229-1231, 1985.
Huub et al., Plant Molec. Biol., 21:985, 1993.
Jones et al., Science, 266:789, 1994.
Kartha et al., Physiol. Plant, 31:217-220, 1974.
Kirihara et al., Mol. Gen. Genet., 211:477-484, 1988.
Klee et al., Bio. Tech., 3(7):637-642, 1985.
Knutzon et al., Proc. Natl. Acad. Sci. USA, 89:2624, 1992.
Lazzeri, Methods Mol. Biol., 49:95-106, 1995.
Lee et al., EMBO J., 7:1241, 1988.
Logemann et al., Bio/technology, 10:305, 1992.
Maliga, Ann. Rev. Plant Biology, 55:289-313, 2004.
Marcotte and Bayley, Nature, 335(6189):454-457, 1988.
Marshall et al., Theor. App. Genet., 83:435, 1992.
Martin et al., Science, 262:1432, 1993.
McDonough et al., J. Biol. Chem., 267(9):5931-5936, 1992.
Miki et al., In: Methods in Plant Molecular Biology and Biotechnology, Glick and Thompson (Eds.), CRC Press, 67-88, 1993.
Miki et al., Theor. App. Genet., 80:449, 1990.
Miles et al., Gene, 32:41-48, 1984.
Mindrinos et al., Cell, 78:1089, 1994.
Narasimhulu, Plant Cell Reports, 7:104-106, 1998.
Ogura, Mem. Fac. Agric. Kagoshima Univ., 6:39-78, 1968.
Ohlsson and Eriksson, Hereditas, 108:173-177, 1988.
Omirulleh et al., Plant Mol. Biol., 21(3):415-428, 1993.
PCT Appln. US93/06487
PCT Appln. WO 91/13972
PCT Appln. WO 92/05251
PCT Appln. WO 92/17598
PCT Appln. WO97/02737
PCT Appln. WO98/27806.
Pelletier et al., Mol. Gen. Genet., 191:244-250, 1983.
Pen et al., BioTechnology, 10:292, 1992.
Poehlman and Sleper, In: Breeding Field Crops, Iowa State University Press, Ames, 1995.
Potrykus et al., Mol. Gen. Genet., 199(2):169-177, 1985.
Przibilla et al., Plant Cell, 3:169, 1991.
Raboy et al., Maydica, 35:383, 1990.
Radke et al., Plant Cell Reports, 11:499-505, 1992.
Radke et al., Theor. Appl. Genet., 75:685-694, 1988.
Rajasekaran et al., Mol. Breed., 2:307-319, 1996.
Rakoczy-Trojanowska, Cell Mol. Biol. Lett., 7(3):849-858, 2002.
Reddy et al. Plant Mol. Biol., 22(2):293-300, 1993.
Sergaard et al., J. Biol. Chem., 268:22480, 1993.
Shiroza et al., J. Bacteol., 170:810, 1988.
Simmonds, In: Principles of crop improvement, Longman, Inc., NY, 369-399, 1979.
Slade et al., Nature Biotechnology, 23:75-81, 2004.
Sneep and Hendriksen, In: Plant breeding perspectives, Wageningen (Ed), Center for Agricultural Publishing and Documentation, 1979.
Sprague and Dudley, In: Corn and Improvement, 3rd ed., 1988.
Steinmetz et al., Mol. Gen. Genet., 20:220, 1985.
Sumitani et al., Biosci. Biotech. Biochem., 57:1243, 1993.
Swanson, Methods in Molecular Biology, 6(17):159, 1990.
Tavladoraki et al., Nature, 366:469, 1993.
Taylor et al., Seventh Int'l Symposium on Molecular Plant-Microbe Interactions (Edinburgh, Scotland) Abstract #497, 1994.
Thompson, Adv. Applied Biol., 7:1-104, 1983.
Uchimiya et al., Mol. Gen. Genet., 204(2):204-207, 1986.
Van Damme et al., Plant Molec. Biol., 24:25, 1994.
Van Hartingsveldt et al., Gene, 127:87, 1993.
Wang et al., Science, 280:1077-1082, 1998.
Ward et. al., In: Oilseed Rape, Farming Press Ltd., Wharefedale Road, Ipswich, Suffolk, 1985.
Williams et al., Nucleic Acids Res., 18:6531-6535, 1990.
Zhang et al., Bio/Technology, 9:996, 1994.

What is claimed is:

1. A seed of canola variety SW 013186, wherein a sample of seed of the variety has been deposited under ATCC Accession No. ______.

2. A plant produced by growing the seed of claim 1.

3. A plant part of the plant of claim 2.

4. The plant part of claim 3, further defined as pollen, an ovule or a cell.

5. A canola plant having all of the physiological and morphological characteristics of the plant of claim 2.

6. A tissue culture of regenerable cells of canola variety SW 013186, wherein the tissue culture regenerates canola plants expressing all the physiological and morphological characteristics of the canola variety SW 013186, and wherein a sample of seed of canola variety SW 013186 has been deposited under ATCC Accession No. ______.

7. The tissue culture of claim 6, wherein the regenerable cells are from embryos, meristematic cells, pollen, leaves, roots, root tips, anther, pistil, flower, seed or stem.

8. A canola plant regenerated from the tissue culture of claim 6, wherein the regenerated canola plant expresses all the physiological and morphological characteristics of the canola variety SW 013186, and wherein a sample of seed of canola variety SW 013186 has been deposited under ATCC Accession No. ______.

9. A method of producing canola seed, comprising crossing a plant of canola variety SW 013186 with itself or a second canola plant, wherein a sample of the seed of said canola variety SW 013186 has been deposited under ATCC Accession No. ______.

10. The method of claim 9, further defined as a method of preparing hybrid canola seed, comprising crossing a plant of canola variety SW 013186 to a second, distinct canola plant, wherein a sample of seed of canola variety SW 013186 has been deposited under ATCC Accession No. ______.

11. A plant produced by the method of claim 10.

12. A method of producing a plant of canola variety SW 013186 further comprising a desired trait, wherein the method comprises introducing a transgene conferring the desired trait into a plant of canola variety SW 013186; wherein the desired trait is selected from the group consisting of male sterility, herbicide resistance, insect or pest resistance, disease resistance, modified seed oil composition, modified phytate metabolism and modified carbohydrate metabolism; wherein a sample of seed of canola variety SW 013186 has been deposited under ATCC Accession No. ______.

13. A plant made by the method of claim 12, wherein the plant comprises the desired trait and otherwise comprises all of the physiological and morphological characteristics of canola variety SW 013186 listed in Table 1 as determined at the 5% significance level when grown in the same environmental conditions.

14. The plant of claim 13, wherein the desired trait is herbicide resistance and the resistance is conferred to an herbicide selected from the group consisting of: glyphosate, sulfonylurea, imidazalinone, glufosinate, phenoxy proprionic acid, cycloshexone, triazine, benzonitrile and broxynil.

15. The plant of claim 13, wherein the desired trait is pest or insect resistance.

16. The plant of claim 15, wherein the desired trait is insect resistance and the transgene encodes a Bacillus thuringiensis (Bt) endotoxin.

17. A method of introducing a desired trait into canola variety SW 013186 comprising:
(a) crossing plants of variety SW 013186, a representative sample of seed of the variety having been deposited under ATCC Accession No. ______, with plants of another canola variety that comprise a desired trait to produce F1 progeny plants, wherein the desired trait is selected from the group consisting of male sterility, herbicide resistance, insect or pest resistance, disease resistance, modified seed oil composition, modified phytate metabolism and modified carbohydrate metabolism;
(b) selecting F1 progeny plants that have the desired trait;
(c) crossing the selected F1 progeny plants with at least a first plant of variety SW 013186 to produce backcross progeny plants;
(d) selecting for backcross progeny plants that have the desired trait and physiological and morphological characteristics of canola variety SW 013186 listed in Table 1 to produce selected backcross progeny plants; and
(e) repeating steps (c) and (d) one or more times in succession to produce selected second or higher backcross progeny plants that comprise the desired trait and all of the physiological and morphological characteristics of canola variety SW 013186 listed in Table 1 as determined at the 5% significance level when grown in the same environmental conditions.

18. A plant produced by the method of claim 17, wherein the plant has the desired trait and all of the physiological and morphological characteristics of canola variety SW 013186 listed in Table 1, as determined at the 5% significance level when grown in the same environmental conditions.

19. The plant of claim 18, wherein the desired trait is herbicide resistance and the resistance is conferred to an herbicide selected from the group consisting of: glyphosate, sulfonylurea, imidazalinone, glufosinate, phenoxy proprionic acid, cycloshexone, triazine, benzonitrile and broxynil.

20. The plant of claim 18, wherein the desired trait is insect resistance and the insect resistance is conferred by a transgene encoding a Bacillus thuringiensis endotoxin.

21. The plant of claim 18, wherein the desired trait is male sterility and the trait is conferred by a cytoplasmic nucleic acid molecule that confers male sterility.

22. A method of producing an inbred canola plant derived from the canola variety SW 013186, the method comprising the steps of:
(a) preparing a progeny plant derived from canola variety SW 013186 by crossing a plant of the canola variety SW 013186 with a second canola plant, wherein a sample of the seed of the canola variety SW 013186 was deposited under ATCC Accession No. ______;
(b) crossing the progeny plant with itself or a second plant to produce a seed of a progeny plant of a subsequent generation;
(c) growing a progeny plant of a subsequent generation from said seed and crossing the progeny plant of a subsequent generation with itself or a second plant; and
(d) repeating steps (b) and (c) for an addition 3-10 generations to produce an inbred canola plant derived from the canola variety SW 013186.
